(19) United States
(12) Patent Application Publication
(10) Pub. No.: US 2017/0079214 A1
(43) Pub. Date: Mar. 23, 2017

(54) Methods for Mushroom Cultivation

(71) Applicant: Novozymes A/S, Bagsvaerd (DK)

(72) Inventors: Wei Li, Beijing (CN); Yi Cao, Beijing (CN)

(73) Assignee: Novozymes A/S, Bagsvaerd (DK)

(21) Appl. No.: 15/311,690

(22) PCT Filed: May 26, 2015
(86) PCT No.: PCT/CN2015/079818

§ 371 Date: Nov. 16, 2016

(30) Foreign Application Priority Data
May 27, 2014 (CN) PCT/CN2014/078527

(51) Int. Cl. A01G 1/04; C12N 9/42; C12N 9/24; A01G 1/00

(52) U.S. Cl. CPC A01G 1/044; A01G 1/001; C12N 9/2437; C12N 9/2482; C12Y 302/01008; C12Y 302/01004

(57) ABSTRACT

A method for cultivating mushrooms, comprising mixing culture material with an enzyme for degrading or converting cellulosic material; a method for improving the yield of mushrooms and/or biological efficiency of mushroom cultivation and an enzyme composition for mushroom cultivation and uses thereof.

### BACKGROUND OF THE INVENTION

Field of the Invention

The present invention relates to methods for cultivating mushroom and specifically pertains to methods for improving the yield of mushrooms by an enzyme for degrading or converting cellulosic material, and an enzyme composition for mushroom cultivation and uses thereof.

Description of the Related Art

“Mushroom” is a generic term which refers to a fruiting body of a number of species of fungus, in particular those species which are edible. Mushrooms are processed, produced, and consumed in many countries on a large scale. Asia holds the majority of the market share of the global mushroom consumption and is closely followed by North America and Europe. The global mushroom market has shown remarkable growth in the recent years and is also showing attractive market potential for the future.

Lignocellulosic material is normally used as culture material for mushroom cultivation. The conversion of lignocellulosic material has the advantages of the ready availability of large amounts of raw material and the desirability of avoiding burning or land filling the materials. Wood, agricultural residues, herbaceous crops, and municipal solid wastes have been used as the culture material for mushroom cultivation. Effective and sustainable usage of the lignocellulosic material is a key factor for cultivating mushroom in high economic value. However, low biological conversion efficiency limits the yield of mushrooms.

CN101974436A discloses a Penicillium expansum W4 strain, with the deposit No. CGMCC No. 4077. It can be used as a component of the fungal formulation for mushroom cultivation. It can shorten the biological conversion time and meet the need of family workshops for small amount of culture material in a low temperature environment.

It would be advantageous in the art to provide an improved method for growing mushroom, especially an improved method for growing mushroom with high yield.

### SUMMARY OF THE INVENTION

The present invention relates to methods for cultivating mushrooms, comprising: (a) providing a culture material, and

(b) mixing the culture material with an enzyme for degrading or converting cellulosic material.

The present invention also relates to methods for improving the yield of mushrooms and/or biological efficiency of mushroom cultivation, comprising: (a) providing a culture material, and (b) mixing the culture material with an enzyme for degrading or converting cellulosic material.

The present invention further relates to an enzyme composition, preferably an enzyme composition for degrading or converting cellulosic material for mushroom cultivation and uses thereof.

### BRIEF DESCRIPTION OF THE FIGURES

FIG. 1 shows a flowchart of cultivating Volvariella volvacea mushroom.

FIG. 2 shows a flowchart of cultivating Flammulina velutipes mushroom.

FIG. 3 shows a flowchart of cultivating Agaricus bisporus mushroom.

FIG. 4 shows a flowchart of cultivating Pleurotus eryngii mushroom.

### DEFINITIONS

Acetylxylan esterase: The term “acetylxylan esterase” means a carboxylesterase (EC 3.1.1.72) that catalyzes the hydrolysis of acetyl groups from polymeric xylan, acetylated xylose, acetylated glucose, alpha-napthyl acetate, and p-nitrophenyl acetate. For purposes of the present invention, acetylxylan esterase activity is determined using 0.5 mM p-nitrophenylacetate as substrate in 50 mM sodium acetate pH 5.0 containing 0.01% TWEEN™ 20 (polyoxyethylene sorbitan monolaurate). One unit of acetylxylan esterase is defined as the amount of enzyme capable of releasing 1 μmole of p-nitrophenolate anion per minute at pH 5, 25° C.

Alpha-L-arabinofuranosidase: The term “alpha-L-arabinofuranosidase” means an alpha-L-arabinofuranoside arabinofuranohydrolase (EC 3.2.1.55) that catalyzes the hydrolysis of terminal non-reducing alpha-L-arabinofuranoside residues in alpha-L-arabinosides. The enzyme acts on alpha-L-arabinofuranosides, alpha-L-arabinans containing (1,3)- and/or (1,5)-linkages, arabinoxylans, and arabinogalactans. Alpha-L-arabinofuranosidase is also known as arabinosidase, alpha-arabinosidase, alpha-L-arabinosidase, alpha-arabinofuranosidase, polysaccharide alpha-L-arabinofuranosidase, alpha-L-arabinofuranoside hydrolase, L-arabinosidase, or alpha-L-arabinanase. For purposes of the present invention, alpha-L-arabinofuranosidase activity is determined using 5 mg of medium viscosity wheat arabinoxylan (Megazyme International Ireland, Ltd., Bray, Co. Wicklow, Ireland) per ml of 100 mM sodium acetate pH 5 in a total volume of 200 μl for 30 minutes at 40° C. followed by arabinose analysis by AMINEX® HPX-87H column chromatography (Bio-Rad Laboratories, Inc., Hercules, Calif., USA).

Alpha-glucuronidase: The term “alpha-glucuronidase” means an alpha-D-glucosiduronate glucuronohydrolase (EC 3.2.1.139) that catalyzes the hydrolysis of an alpha-D-glucuronoside to D-glucuronate and an alcohol. For purposes of the present invention, alpha-glucuronidase activity is determined according to de Vries, 1998, J. Bacteriol. 180: 243-249. One unit of alpha-glucuronidase equals the amount of enzyme capable of releasing 1 μmole of glucuronic or 4-O-methylglucuronic acid per minute at pH 5, 40° C.

Amylase: The term “amylase” means an enzyme that catalyzes the endo-hydrolysis of starch and other linear and branched oligo- and polysaccharides. It includes alpha-amylase (EC 3.2.1.1), beta-amylase (EC 3.2.1.2), and gamma-amylase (EC 3.2.1.3).

Auxiliary Activity 9: The term “Auxiliary Activity 9” or “AA9” means a polypeptide classified as a lytic polysaccharide monooxygenase (Quinlan et al., 2011, Proc. Natl. Acad. Sci. USA 208: 15079-15084; Phillips et al., 2011, ACS Chem. Biol. 6: 1399-1406; Lin et al., 2012, Structure 20: 1051-1061). AA9 polypeptides were formerly classified into the glycoside hydrolase Family 61 (GH61) according to Henrissat, 1991, Biochem. J. 280: 309-316, and Henrissat and Bairoch, 1996, Biochem. J. 316: 695-696.

AA9 polypeptides enhance the hydrolysis of a cellulosic material by an enzyme having cellulolytic activity. Cellulolytic enhancing activity can be determined by measuring the increase in reducing sugars or the increase of the total of cellobiose and glucose from the hydrolysis of a cellulosic material by cellulolytic enzyme under the following conditions: 1-50 mg of total protein/g of cellulose in pretreated corn stover (PCS), wherein total protein is comprised of 50-99.5% w/w cellulolytic enzyme protein and 0.5-50% w/w protein of an AA9 polypeptide for 1-7 days at a suitable temperature, such as 40° C.-80° C., e.g., 50° C., 55° C., 60° C., 65° C., or 70° C., and a suitable pH, such as 4-9, e.g., 4.5, 5.0, 5.5, 6.0, 6.5, 7.0, 7.5, 8.0, or 8.5, compared to a control hydrolysis with equal total protein loading without cellulolytic enhancing activity (1-50 mg of cellulolytic protein/g of cellulose in PCS).

AA9 polypeptide enhancing activity can be determined using a mixture of CELLUCLAST™ 1.5 L (Novozymes A/S, Bagsværd, Denmark) and beta-glucosidase as the source of the cellulolytic activity, wherein the beta-glucosidase is present at a weight of at least 2-5% protein of the cellulase protein loading. In one aspect, the beta-glucosidase is an Aspergillus oryzae beta-glucosidase (e.g., recombinantly produced in Aspergillus oryzae according to WO 02/095014). In another aspect, the beta-glucosidase is an Aspergillus fumigatus beta-glucosidase (e.g., recombinantly produced in Aspergillus oryzae as described in WO 02/095014).

AA9 polypeptide enhancing activity can also be determined by incubating an AA9 polypeptide with 0.5% phosphoric acid swollen cellulose (PASC), 100 mM sodium acetate pH 5, 1 mM MnSO₄, 0.1% gallic acid, 0.025 mg/ml of Aspergillus fumigatus beta-glucosidase, and 0.01% TRITON® X-100 (441,1,3,3-tetramethylbutyl)phenyl-polyethylene glycol) for 24-96 hours at 40° C. followed by determination of the glucose released from the PASC.

AA9 polypeptide enhancing activity can also be determined according to WO 2013/028928 for high temperature compositions.

AA9 polypeptides enhance the hydrolysis of a cellulosic material catalyzed by enzyme having cellulolytic activity by reducing the amount of cellulolytic enzyme required to reach the same degree of hydrolysis preferably at least 1.01-fold, e.g., at least 1.05-fold, at least 1.10-fold, at least 1.25-fold, at least 1.5-fold, at least 2-fold, at least 3-fold, at least 4-fold, at least 5-fold, at least 10-fold, or at least 20-fold.

Beta-glucosidase: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. For purposes of the present invention, beta-glucosidase activity is determined using p-nitrophenyl-beta-D-glucopyranoside as substrate according to the procedure of Venturi et al., 2002, Extracellular beta-D-glucosidase from Chaetomium thermophilum var. coprophilum: production, purification and some biochemical properties, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20.

Beta-xylosidase: The term “beta-xylosidase” means a beta-D-xyloside xylohydrolase (E.C. 3.2.1.37) that catalyzes the exo-hydrolysis of short beta (1→4)-xylooligosaccharides to remove successive D-xylose residues from non-reducing termini. For purposes of the present invention, one unit of beta-xylosidase is defined as 1.0 μmole of p-nitrophenolate anion produced per minute at 40° C., pH 5 from 1 mM p-nitrophenyl-beta-D-xyloside as substrate in 100 mM sodium citrate containing 0.01% TWEEN® 20.

Catalase: The term “catalase activity” is defined herein as a hydrogen-peroxide:hydrogen-peroxide oxidoreductase activity (EC 1.11.1.6) that catalyzes the conversion of 2 H₂O₂ to O₂+2 H₂O. For purposes of the present invention, catalase activity is determined according to U.S. Pat. No. 5,646,025. One unit of catalase activity equals the amount of enzyme that catalyzes the oxidation of 1 μmole of hydrogen peroxide under the assay conditions.

Cellobiohydrolase: The term “cellobiohydrolase” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing or non-reducing ends of the chain (Teen, 1997, Crystalline cellulose degradation: New insight into the function of cellobiohydrolases, Trends in Biotechnology 15: 160-167; Teen et al., 1998, Trichoderma reesei cellobiohydrolases: why so efficient on crystalline cellulose?, Biochem. Soc. Trans. 26: 173-178). Cellobiohydrolase activity is determined according to the procedures described by Lever et al., 1972, Anal. Biochem. 47: 273-279; van Tilbeurgh et al., 1982, FEBS Letters, 149: 152-156; van Tilbeurgh and Claeyssens, 1985, FEBS Letters, 187: 283-288; and Tomme et al., 1988, Eur. J. Biochem. 170: 575-581. In the present invention, the Tomme et al. method can be used to determine cellobiohydrolase activity.

Cellulolytic enzyme or cellulase: The term “cellulolytic enzyme” or “cellulase” means one or more (e.g., several) enzymes that hydrolyze a cellulosic material. Such enzymes include endoglucanase(s), cellobiohydrolase(s), beta-glucosidase(s), or combinations thereof. The two basic approaches for measuring cellulolytic activity include: (1) measuring the total cellulolytic activity, and (2) measuring the individual cellulolytic activities (endoglucanases, cellobiohydrolases, and beta-glucosidases) as reviewed in Zhang et al., Outlook for cellulase improvement: Screening and selection strategies, 2006, Biotechnology Advances 24: 452-481. Total cellulolytic activity is usually measured using insoluble substrates, including Whatman No 1 filter paper, microcrystalline cellulose, bacterial cellulose, algal cellulose, cotton, pretreated lignocellulose, etc. The most common total cellulolytic activity assay is the filter paper assay using Whatman No 1 filter paper as the substrate. The assay was established by the International Union of Pure and Applied Chemistry (IUPAC) (Ghose, 1987, Measurement of cellulase activities, Pure Appl. Chem. 59: 257-68).

For purposes of the present invention, cellulolytic enzyme activity is determined by measuring the increase in hydrolysis of a cellulosic material by cellulolytic enzyme(s) under the following conditions: 1-50 mg of cellulolytic enzyme protein/g of cellulose in PCS (or other pretreated cellulosic material) for 3-7 days at a suitable temperature, e.g., 50° C., 55° C., or 60° C., compared to a control hydrolysis without addition of cellulolytic enzyme protein. Typical conditions are 1 ml reactions, washed or unwashed PCS, 5% insoluble solids, 50 mM sodium acetate pH 5, 1 mM MnSO₄, 50° C., 55° C., or 60° C., 72 hours, sugar analysis by AMINEX® HPX-87H column (Bio-Rad Laboratories, Inc., Hercules, Calif., USA).

Endoglucanase: The term “endoglucanase” means an endo-1,4-(1,3;1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). For purposes of the present invention, endoglucanase activity is determined using carboxymethyl cellulose (CMC) as substrate according to the procedure of Ghose, 1987, Pure and Appl. Chem. 59: 257-268, at pH 5, 40° C.

Feruloyl esterase: The term “feruloyl esterase” means a 4-hydroxy-3-methoxycinnamoyl-sugar hydrolase (EC 3.1.1.73) that catalyzes the hydrolysis of 4-hydroxy-3-methoxycinnamoyl (feruloyl) groups from esterified sugar, which is usually arabinose in “natural” substrates, to produce ferulate (4-hydroxy-3-methoxycinnamate) Feruloyl esterase is also known as ferulic acid esterase, hydroxycinnamoyl esterase, FAE-III, cinnamoyl ester hydrolase, FAEA, cinnAE, FAE-I, or FAE-II. For purposes of the present invention, feruloyl esterase activity is determined using 0.5 mM p-nitrophenylferulate as substrate in 50 mM sodium acetate pH 5.0. One unit of feruloyl esterase equals the amount of enzyme capable of releasing 1 μmole of p-nitrophenolate anion per minute at pH 5, 25° C.

Hemicellulolytic enzyme or hemicellulase: The term “hemicellulolytic enzyme” or “hemicellulase” means one or more (e.g., several) enzymes that hydrolyze a hemicellulosic material. See, for example, Shallom, D. and Shoham, Y. Microbial hemicellulases. Current Opinion In Microbiology, 2003, 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase. The substrates of these enzymes, the hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, or carbohydrate esterases (CEs), which hydrolyze ester linkages of acetate or ferulic acid side groups. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59: 1739-1752, at a suitable temperature, e.g., 50° C., 55° C., or 60° C., and pH, e.g., 5.0 or 5.5.

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. For purposes of the present invention, xylanase activity is determined with 0.2% AZCL-arabinoxylan as substrate in 0.01% TRITON® X-100 and 200 mM sodium phosphate buffer pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6 buffer.

### DETAILED DESCRIPTION OF THE INVENTION

Methods of Cultivating Mushroom

The present invention relates to methods for cultivating mushrooms, comprising: (a) providing a culture material, and (b) mixing the culture material with an enzyme for degrading or converting cellulosic material.

The methods for cultivating mushroom is known in the art, see for example, Chang S T, Yau C K. A simple technique for indoor cultivation of straw mushroom, Mushroom news, 1970, 18:9-11; and Chang S, Miles P G. Edible mushrooms and their cultivation. Edible mushrooms and their cultivation 1989, 345. Normally, the production of the mushroom spawn for inoculation is one of the initial stages for mushroom cultivation. Typically, this consists of the preparation of a large number of kernels or hulls. The kernels or hulls are composted (and sometimes sterilized for wood rot mushroom) and then innoculated with the mycelia of the particular species of mushroom desired. The mushroom mycelia are then allowed to proliferate upon the kernels or hulls until the individual kernels or hulls are completely covered by the living mushroom tissue. The mushroom mycelia-covered kernels or hulls which result are known in the industry as spawn.

The planting of the spawn is the second stage of mushroom growing. Culture material in which the spawn is to be planted is prepared, and aged to the proper stage, under proper temperature and environmental conditions. Culture material traditionally comprises the cleanings from horse stables or other similar composts, although modern culture material comes from a variety of sources. It is necessary to select and treat the culture material carefully so that it has good nutrient content and does not contain undue amounts of acid or various chemical and biological inhibitors such as high ammonia content. High concentrations of chemicals such as acids or ammonia will hinder the growth of the mushrooms and reduce the efficiency of the operation. The actual planting consists of distributing the spawn throughout the bed filled with the culture material in such a manner that the culture material contained in the bed is reasonably accessible to each spawn. To achieve this, the spawns are typically evenly distributed over the culture material surface and then mechanically mixed into the culture material.

Once the spawn has been planted, it is allowed to vegetate and grow under controlled environmental conditions until it is “cased” and then continues to vegetate until the mycelia are ready for fruition. The amount of time necessary for such vegetative growth is dependent on the precise environmental conditions, the particular type of mushroom and the nutritive content of the culture material bed.

After the vegetative phase has continued for the appropriate length of time, an operation known as casing is performed for some mushrooms. Casing involves spreading a thin layer of soil over the culture material bed. This soil is kept moist. The bed temperature is thus reduced for a short period. This temperature reduction has the effect of causing the mushroom mycelia to fruit or “crop” and thus send up the actual mushrooms through the casing soil.

The final stage of a mushroom crop is the actual fruition or “cropping”. During this stage the mature fungus sends up the fruiting bodies which are marketed as mushrooms. Each particular colony of fungus will send up fruit when it has reached the proper stage. The actual time frame of the fruition varies throughout the bed.

Based on the culture materials needed for mushroom growing, mushroom can be roughly divided into wood rot fungi and grass rot fungi. Wood rot mushroom, which normally uses forestries or woods as the main nutrients, includes but is not limited to Pleurotus eryngii, Lentinula edodes, Auricularia auricular, Pleurotus ostreatus, Ganoderma Lucidum and Hericium erinaceus. Grass rot mushroom normally uptakes the organic matter in the rot grass for example, rice straw or wheat straw, as main nutrients. Grass rot mushroom includes, but is not limited to Agaricus bisporus, Volvariella volvacea, and Copyinds comatus. Grass rot mushroom mycelia can grow on sucrose and glucose and rot agricultural residue, livestock and poultry manure and the fungal residue of wood rot fungi, as carbon source.

In a preferable aspect, the methods of the present invention comprise

(a) providing a culture material,

(b) mixing the culture material with an enzyme composition comprising cellulase and hemicellulase.

In a preferable aspect, the methods of the present invention comprise

(a) providing a culture material,

(b) mixing the culture material with cellulase,

(c) mixing the culture material with hemicellulase,

wherein step (b) and step (c) is carried out simultaneously or sequentially.

In a preferable aspect, the present invention relates to methods for mushroom cultivation, comprising

(a) providing a culture material,

(b) mixing the culture material with an enzyme, and

(c) subjecting the mixed culture material to composting.

In another preferable aspect, the present methods further comprise subjecting the composted culture material to a second composting.

In a preferable aspect, the methods of the present invention comprise

inoculating the culture material with mushroom mycelia and allowing the mushroom mycelia to develop into mushroom; and optionally recovering the mushroom.

In another aspect, the present invention relates to methods for improving the yield of mushrooms and/or biological efficiency of mushroom cultivation, comprising: (a) providing a culture material, and (b) mixing the culture material with an enzyme for degrading or converting cellulosic material.

In a preferable aspect, the present invention relates to methods for improving the yield of mushrooms and/or biological efficiency of mushroom cultivation, comprising

(a) providing a culture material,

(b) mixing the culture material with an enzyme composition comprising cellulase and hemicellulase.

In a preferable aspect, the present invention relates to methods for improving the yield of mushrooms and/or biological efficiency of mushroom cultivation, comprising

(a) providing a culture material,

(b) mixing the culture material with cellulase,

(c) mixing the culture material with hemicellulase,

wherein step (b) and step (c) is carried out simultaneously or sequentially.

In a preferable aspect, the present invention relates to use of a cellulase and a hemicellulose for mushroom cultivation.

In a preferable aspect, the present invention relates to use of a cellulase and a hemicellulose for improvement of yield of mushroom and/or biological efficiency of mushroom cultivation.

Culture Material for Mushroom

The culture material for mushroom is dependent on the varieties of mushroom. But normally, the culture material comprises cellulosic material.

The term “cellulosic material” means any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1→4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Cellulose is generally found, for example, in the stems, leaves, hulls, husks, and cobs of plants or leaves, branches, bran, and wood of trees. The cellulosic material can be, but is not limited to, agricultural residue, herbaceous material (including energy crops), municipal solid waste, pulp and paper mill residue, waste paper, and wood (including forestry residue) (see, for example, Wiselogel et al., 1995, in Handbook on Bioethanol (Charles E. Wyman, editor), pp. 105-118, Taylor & Francis, Washington D.C.; Wyman, 1994, Bioresource Technology 50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, New York). It is understood herein that the cellulose may be in the form of lignocellulose, a plant cell wall material containing lignin, cellulose, and hemicellulose in a mixed matrix. In a preferred aspect, the cellulosic material is any biomass material. In another preferred aspect, the cellulosic material is lignocellulose, which comprises cellulose, hemicelluloses, and lignin.

In one aspect, the cellulosic material is agricultural residue. In another aspect, the cellulosic material is herbaceous material (including energy crops). In another aspect, the cellulosic material is municipal solid waste. In another aspect, the cellulosic material is pulp and paper mill residue. In another aspect, the cellulosic material is waste paper. In another aspect, the cellulosic material is wood (including forestry residue).

In another aspect, the cellulosic material is arundo. In another aspect, the cellulosic material is bagasse. In another aspect, the cellulosic material is bamboo. In another aspect, the cellulosic material is corn cob. In another aspect, the cellulosic material is corn fiber. In another aspect, the cellulosic material is corn stover. In another aspect, the cellulosic material is miscanthus. In another aspect, the cellulosic material is orange peel. In another aspect, the cellulosic material is rice straw. In another aspect, the cellulosic material is switchgrass. In another aspect, the cellulosic material is wheat straw.

In another aspect, the cellulosic material is aspen. In another aspect, the cellulosic material is eucalyptus. In another aspect, the cellulosic material is fir. In another aspect, the cellulosic material is pine. In another aspect, the cellulosic material is poplar. In another aspect, the cellulosic material is spruce. In another aspect, the cellulosic material is willow.

In another aspect, the cellulosic material is algal cellulose. In another aspect, the cellulosic material is bacterial cellulose. In another aspect, the cellulosic material is cotton waste. In another aspect, the cellulosic material is cotton linter. In another aspect, the cellulosic material is filter paper. In another aspect, the cellulosic material is saw dust. In another aspect, the cellulosic material is hull of cotton seed. In another aspect, the cellulosic material is kernel of grains.

In another aspect, the cellulosic material is an aquatic biomass. As used herein the term “aquatic biomass” means biomass produced in an aquatic environment by a photosynthesis process. The aquatic biomass can be algae, emergent plants, floating-leaf plants, or submerged plants.

In another preferable aspect, the cellulosic material can be selected from the group consisting of agricultural residue, herbaceous material, municipal solid waste, pulp and paper mill residue, waste paper, bran and wood; preferably, arundo, bagasse, bamboo, corn cob, corn fiber, corn stover, miscanthus, orange peel, rice straw, switchgrass, wheat straw, eucalyptus, fir, pine, poplar, spruce, willow, algal cellulose, bacterial cellulose, cotton waste, cotton linter, saw dust, hull of cotton seed, kernel of grains, and filter paper.

The cellulosic material may be used as is or may be subjected to pretreatment, using conventional methods known in the art. In a preferred aspect, the cellulosic material is subjected to “mechanical pretreatment” before it is mixed with an enzyme, according to the present invention. “Mechanical pretreatment” refers to various types of grinding or milling (e.g., dry milling, wet milling, or vibratory ball milling).

In addition to the cellulosic material, the culture material can comprises any matters which are good to the growth of mushroom, for example, manure, starch, gypsum, sugar, urea, calcium superphosphate, calcium carbonate and/or lime.

Enzyme and Enzyme Composition

The enzyme compositions can comprise any protein that is useful in degrading or converting cellulosic material. The compositions may comprise one enzyme as the major enzymatic component, e.g., a monocomponent composition, or multiple enzymes. The compositions may be prepared in accordance with methods known in the art and may be in the form of a liquid or a dry composition. The compositions may be stabilized in accordance with methods known in the art.

In one aspect, an enzyme for degrading or converting a cellulosic material comprises one or more (e.g., several) enzymes having cellulolytic and/or hemicellulolytic activity.

In an embodiment, the enzyme comprises a cellulase and further comprises one or more (e.g., several) proteins selected from the group consisting of a AA9 polypeptide having cellulolytic enhancing activity, a hemicellulase, an amylase, an esterase, an expansin, a catalase, a laccase, a ligninolytic enzyme, a pectinase, a peroxidase, a protease, and a swollenin. In another aspect, the cellulase is preferably one or more (e.g., several) enzymes selected from the group consisting of an endoglucanase, a cellobiohydrolase, and a beta-glucosidase. In another aspect, the hemicellulase is preferably one or more (e.g., several) enzymes selected from the group consisting of an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase.

In another embodiment, the enzyme composition comprises one or more (e.g., several) cellulolytic enzymes. In another aspect, the enzyme composition comprises or further comprises one or more (e.g., several) hemicellulolytic enzymes. In another aspect, the enzyme composition comprises one or more (e.g., several) cellulolytic enzymes and one or more (e.g., several) hemicellulolytic enzymes. In another aspect, the enzyme composition comprises one or more (e.g., several) enzymes selected from the group of cellulolytic enzymes and hemicellulolytic enzymes. In another aspect, the enzyme composition comprises an endoglucanase. In another aspect, the enzyme composition comprises a cellobiohydrolase. In another aspect, the enzyme composition comprises a beta-glucosidase. In another aspect, the enzyme composition comprises a polypeptide having cellulolytic enhancing activity. In another aspect, the enzyme composition comprises an endoglucanase and a polypeptide having cellulolytic enhancing activity. In another aspect, the enzyme composition comprises a cellobiohydrolase and a polypeptide having cellulolytic enhancing activity. In another aspect, the enzyme composition comprises a beta-glucosidase and a polypeptide having cellulolytic enhancing activity. In another aspect, the enzyme composition comprises an endoglucanase and a cellobiohydrolase. In another aspect, the enzyme composition comprises an endoglucanase and a beta-glucosidase. In another aspect, the enzyme composition comprises a cellobiohydrolase and a beta-glucosidase. In another aspect, the enzyme composition comprises an endoglucanase, a cellobiohydrolase, and a polypeptide having cellulolytic enhancing activity. In another aspect, the enzyme composition comprises an endoglucanase, a beta-glucosidase, and a polypeptide having cellulolytic enhancing activity. In another aspect, the enzyme composition comprises a cellobiohydrolase, a beta-glucosidase, and a polypeptide having cellulolytic enhancing activity. In another aspect, the enzyme composition comprises an endoglucanase, a cellobiohydrolase, and a beta-glucosidase. In another aspect, the enzyme composition comprises an endoglucanase, a cellobiohydrolase, a beta-glucosidase, and a polypeptide having cellulolytic enhancing activity.

In another embodiment, the enzyme composition comprises an acetylmannan esterase. In another aspect, the enzyme composition comprises an acetylxylan esterase. In another aspect, the enzyme composition comprises an arabinanase (e.g., alpha-L-arabinanase). In another aspect, the enzyme composition comprises an arabinofuranosidase (e.g., alpha-L-arabinofuranosidase). In another aspect, the enzyme composition comprises a coumaric acid esterase. In another aspect, the enzyme composition comprises a feruloyl esterase. In another aspect, the enzyme composition comprises a galactosidase (e.g., alpha-galactosidase and/or beta-galactosidase). In another aspect, the enzyme composition comprises a glucuronidase (e.g., alpha-D-glucuronidase). In another aspect, the enzyme composition comprises a glucuronoyl esterase. In another aspect, the enzyme composition comprises a mannanase. In another aspect, the enzyme composition comprises a mannosidase (e.g., beta-mannosidase). In another aspect, the enzyme composition comprises a xylanase. In a preferred aspect, the xylanase is a Family 10 xylanase. In another aspect, the enzyme composition comprises a xylosidase (e.g., beta-xylosidase).

In another embodiment, the enzyme composition comprises an esterase. In another aspect, the enzyme composition comprises an expansin. In another aspect, the enzyme composition comprises a catalase. In another aspect, the enzyme composition comprises a laccase. In another aspect, the enzyme composition comprises a ligninolytic enzyme. In a preferred aspect, the ligninolytic enzyme is a manganese peroxidase. In another preferred aspect, the ligninolytic enzyme is a lignin peroxidase. In another preferred aspect, the ligninolytic enzyme is a H₂O₂-producing enzyme. In another aspect, the enzyme composition comprises a pectinase. In another aspect, the enzyme composition comprises a peroxidase. In another aspect, the enzyme composition comprises a protease. In another aspect, the enzyme composition comprises a swollenin. In a preferable aspect, the enzyme composition comprises acid cellulase (including acid endocellulase, and acid exocellulase). In a preferable aspect, the enzyme composition comprises acid cellulase (including acid endocellulase, and acid exocellulase), and beta-glucosidase. In another preferable aspect, the enzyme composition comprises acid cellulase, neutral cellulase, and beta-glucosidase. In another preferable aspect, the enzyme composition comprises acid cellulose, acid hemicellulase (including endoxylanase) endocellulase, exocellulase and beta-glucosidas. In another preferable aspect, the enzyme composition comprises acid cellulase, neutral cellulose, acid hemicellulase (including endoxylanase) endocellulase, exocellulase and beta-glucosidas.

In the methods of the present invention, the enzyme(s) can be added prior to or during composting or inoculation. The enzymes having cellulolytic and the enzymes having hemicellulolytic activity can be added simultaneously or sequentially.

One or more (e.g., several) components of the enzyme composition may be wild-type proteins, recombinant proteins, or a combination of wild-type proteins and recombinant proteins. For example, one or more (e.g., several) components may be native proteins of a cell, which is used as a host cell to express recombinantly one or more (e.g., several) other components of the enzyme composition. One or more (e.g., several) components of the enzyme composition may be produced as monocomponents, which are then combined to form the enzyme composition. The enzyme composition may be a combination of multicomponent and monocomponent protein preparations.

The enzymes used in the methods of the present invention may be in any form suitable for use, such as, for example, a fermentation broth formulation or a cell composition, a cell lysate with or without cellular debris, a semi-purified or purified enzyme preparation, or a host cell as a source of the enzymes. The enzyme may be a dry powder or granulate, a non-dusting granulate, a liquid, a stabilized liquid, or a stabilized protected enzyme. Liquid enzyme preparations may, for instance, be stabilized by adding stabilizers such as a sugar, a sugar alcohol or another polyol, and/or lactic acid or another organic acid according to established processes.

The optimum amounts of the enzymes depend on several factors including, but not limited to, the mixture of component cellulolytic enzymes, the cellulosic material, the concentration of cellulosic material in the culture material, the pretreatment(s) of the cellulosic material, temperature, time, and pH.

In a preferred aspect, an effective amount of enzyme to the culture material is about 0.005 to about 100 mg, preferably about 0.01 to about 50 mg, more preferably about 0.05 to about 25 mg, more preferably about 0.1 to about 20 mg, more preferably about 0.1 to about 15 mg, even more preferably about 0.1 to about 10 mg, and most preferably about 0.5 to about 10 mg enzyme protein per g of the culture material.

In a preferred aspect, an effective amount of cellulase to the culture material is about 0.005 to about 50 mg, preferably about 0.01 to about 40 mg, more preferably about 0.05 to about 25 mg, more preferably about 0.1 to about 20 mg, more preferably about 0.25 to about 15 mg, even more preferably about 0.5 to about 10 mg, and most preferably about 0.5 to about 5 mg cellulase per g of the culture material.

In a preferred aspect, an effective amount of hemicellulase to the culture material is about 0.001 to about 50 mg, preferably about 0.005 to about 40 mg, more preferably about 0.01 to about 25 mg, more preferably about 0.05 to about 20 mg, more preferably about 0.05 to about 15 mg, even more preferably about 0.075 to about 10 mg, and most preferably about 0.1 to about 5 mg hemicellulase per g of the culture material.

In another preferred aspect, an effective amount of hemicellulase to cellulase is about 0.005 to about 1.0 g, preferably about 0.005 to about 0.75 g, more preferably about 0.01 to about 0.75 g, more preferably about 0.05 to about 0.5 g, more preferably about 0.075 to about 0.5 g, even more preferably about 0.1 to about 0.5 g, and most preferably about 0.1 to about 0.3 g hemicellulase per g of cellulase.

The polypeptides having cellulolytic enzyme activity or hemicellulolytic enzyme activity as well as other proteins/polypeptides useful in the degradation of the cellulosic material, e.g., polypeptides having cellulolytic enhancing activity (collectively hereinafter “polypeptides having enzyme activity”) can be derived or obtained from any suitable origin, including, bacterial, fungal, yeast, plant, or mammalian origin. The term “obtained” also means herein that the enzyme may have been produced recombinantly in a host organism employing methods described herein, wherein the recombinantly produced enzyme is either native or foreign to the host organism or has a modified amino acid sequence, e.g., having one or more (e.g., several) amino acids that are deleted, inserted and/or substituted, i.e., a recombinantly produced enzyme that is a mutant and/or a fragment of a native amino acid sequence or an enzyme produced by nucleic acid shuffling processes known in the art. Encompassed within the meaning of a native enzyme are natural variants and within the meaning of a foreign enzyme are variants obtained recombinantly, such as by site-directed mutagenesis or shuffling.

A polypeptide having enzyme activity may be a bacterial polypeptide. For example, the polypeptide may be a gram positive bacterial polypeptide such as a Bacillus, Streptococcus, Streptomyces, Staphylococcus, Enterococcus, Lactobacillus, Lactococcus, Clostridium, Geobacillus, Caldicellulosiruptor, Acidothermus, Thermobifidia, or Oceanobacillus polypeptide having enzyme activity, or a Gram negative bacterial polypeptide such as an E. coli, Pseudomonas, Salmonella, Campylobacter, Helicobacter, Flavobacterium, Fusobacterium, Ilyobacter, Neisseria, or Ureaplasma polypeptide having enzyme activity.

In one aspect, the polypeptide is a Bacillus alkalophilus, Bacillus amyloliquefaciens, Bacillus brevis, Bacillus circulars, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis polypeptide having enzyme activity.

In another aspect, the polypeptide is a Streptococcus equisimilis, Streptococcus pyogenes, Streptococcus uberis, or Streptococcus equi subsp. Zooepidemicus polypeptide having enzyme activity.

In another aspect, the polypeptide is a Streptomyces achromogenes, Streptomyces avermitilis, Streptomyces coelicolor, Streptomyces griseus, or Streptomyces lividans polypeptide having enzyme activity.

The polypeptide having enzyme activity may also be a fungal polypeptide, and more preferably a yeast polypeptide such as a Candida, Kluyveromyces, Pichia, Saccharomyces, Schizosaccharomyces, or Yarrowia polypeptide having enzyme activity; or more preferably a filamentous fungal polypeptide such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria polypeptide having enzyme activity.

In one aspect, the polypeptide is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis polypeptide having enzyme activity.

In one aspect, the polypeptide is an Acremonium cellulolyticus, Aspergillus aculeatus, Aspergillus awamori, Aspergillus fumigatus, Aspergillus foetidus, Aspergillus japonicus, Aspergillus nidulans, Aspergillus niger, Aspergillus oryzae, Chrysosporium keratinophilum, Chrysosporium lucknowense, Chrysosporium tropicum, Chrysosporium merdarium, Chrysosporium inops, Chrysosporium pannicola, Chrysosporium queenslandicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia spededonium, Thielavia setosa, Thielavia subthermophila, Thielavia terrestris, Trichoderma harzianum, Trichoderma koningii, Trichoderma longibrachiatum, Trichoderma reesei, Trichoderma viride, or Trichophaea saccata polypeptide having enzyme activity.

Chemically modified or protein engineered mutants of polypeptides having enzyme activity may also be used.

One or more (e.g., several) components of the enzyme composition may be a recombinant component, i.e., produced by cloning of a DNA sequence encoding the single component and subsequent cell transformed with the DNA sequence and expressed in a host (see, for example, WO 91/17243 and WO 91/17244). The host is preferably a heterologous host (enzyme is foreign to host), but the host may under certain conditions also be a homologous host (enzyme is native to host). Monocomponent cellulolytic proteins may also be prepared by purifying such a protein from a fermentation broth.

In one aspect, the one or more (e.g., several) cellulolytic enzymes comprise a commercial cellulolytic enzyme preparation. Examples of commercial cellulolytic enzyme preparations suitable for use in the present invention include, for example, CELLIC® CTec Ctec3 (Novozymes A/S), CELLIC® CTec CTec2 (Novozymes A/S), CELLIC® CTec (Novozymes A/S), CELLUCLAST™ (Novozymes A/S), NOVOZYM™ 188 (Novozymes A/S), CELLUZYME™ (Novozymes A/S), CEREFLO™ (Novozymes A/S), and ULTRAFLO™ (Novozymes A/S), ACCELERASE™ (Genencor Int.), LAMINEX™ (Genencor Int.), SPEZYME™ CP (Genencor Int.), FILTRASE® NL (DSM); METHAPLUS® S/L 100 (DSM), ROHAMENT™ 7069 W (Röhm GmbH), FIBREZYME® LDI (Dyadic International, Inc.), FIBREZYME® LBR (Dyadic International, Inc.), or VISCOSTAR® 150 L (Dyadic International, Inc.), CAREZYME® (Novozymes A/S), CELLUCLEAN® (Novozymes A/S), Renozyme® (Novozymes A/S), and Puradax®, Puradax HA and Puradax EG (Genencor).

Examples of bacterial endoglucanases that can be used in the method of the present invention, include, but are not limited to, an Acidothermus cellulolyticus endoglucanase (WO 91/05039; WO 93/15186; U.S. Pat. No. 5,275,944; WO 96/02551; U.S. Pat. No. 5,536,655, WO 00/70031, WO 05/093050); Thermobifida fusca endoglucanase III (WO 05/093050); and Thermobifida fusca endoglucanase V (WO 05/093050).

Examples of fungal endoglucanases that can be used in the present invention, include, but are not limited to, a Trichoderma reesei endoglucanase I (Penttila et al., 1986, Gene 45: 253-263; Trichoderma reesei Cel7B endoglucanase I (GENBANK™ accession no. M15665); Trichoderma reesei endoglucanase II (Saloheimo, et al., 1988, Gene 63:11-22; Trichoderma reesei Cel5A endoglucanase II (GENBANK™ accession no. M19373); Trichoderma reesei endoglucanase III (Okada et al., 1988, Appl. Environ. Microbiol. 64: 555-563; GENBANK™ accession no. AB003694); Trichoderma reesei endoglucanase V (Saloheimo et al., 1994, Molecular Microbiology 13: 219-228; GENBANK™ accession no. Z33381); Aspergillus aculeatus endoglucanase (Ooi et al., 1990, Nucleic Acids Research 18: 5884); Aspergillus kawachii endoglucanase (Sakamoto et al., 1995, Current Genetics 27: 435-439); Erwinia carotovara endoglucanase (Saarilahti et al., 1990, Gene 90: 9-14); Fusarium oxysporum endoglucanase (GENBANK™ accession no. L29381); Humicola grisea var. thermoidea endoglucanase (GENBANK™ accession no. AB003107); Melanocarpus albomyces endoglucanase (GENBANK™ accession no. MAL515703); Neurospora crassa endoglucanase (GENBANK™ accession no. XM_324477); Humicola insolens endoglucanase V; Myceliophthora thermophila CBS 117.65 endoglucanase; basidiomycete CBS 495.95 endoglucanase; basidiomycete CBS 494.95 endoglucanase; Thielavia terrestris NRRL 8126 CEL6B endoglucanase; Thielavia terrestris NRRL 8126 CEL6C endoglucanase; Thielavia terrestris NRRL 8126 CEL7C endoglucanase; Thielavia terrestris NRRL 8126 CEL7E endoglucanase; Thielavia terrestris NRRL 8126 CEL7F endoglucanase; Cladorrhinum foecundissimum ATCC 62373 CEL7A endoglucanase; and Trichoderma reesei strain No. VTT-D-80133 endoglucanase (GENBANK™ accession no. M15665).

Examples of cellobiohydrolases useful in the present invention include, but are not limited to, Trichoderma reesei cellobiohydrolase I; Trichoderma reesei cellobiohydrolase II; Humicola insolens cellobiohydrolase I; Myceliophthora thermophila cellobiohydrolase II; Thielavia terrestris cellobiohydrolase II (CEL6A); Chaetomium thermophilum cellobiohydrolase I; and Chaetomium thermophilum cellobiohydrolase II.

Examples of beta-glucosidases useful in the present invention include, but are not limited to, Aspergillus oryzae beta-glucosidase; Aspergillus fumigatus beta-glucosidase; Penicillium brasilianum IBT 20888 beta-glucosidase; Aspergillus niger beta-glucosidase; and Aspergillus aculeatus beta-glucosidase.

The Aspergillus oryzae beta-glucosidase can be obtained according to WO 2002/095014. The Aspergillus fumigatus beta-glucosidase can be obtained according to WO 2005/047499. The Penicillium brasilianum beta-glucosidase can be obtained according to WO 2007/019442. The Aspergillus niger beta-glucosidase can be obtained according to Dan et al., 2000, J. Biol. Chem. 275: 4973-4980. The Aspergillus aculeatus beta-glucosidase can be obtained according to Kawaguchi et al., 1996, Gene 173: 287-288.

The beta-glucosidase may be a fusion protein. In one aspect, the beta-glucosidase is the Aspergillus oryzae beta-glucosidase variant BG fusion protein or the Aspergillus oryzae beta-glucosidase fusion protein obtained according to WO 2008/057637.

Other useful endoglucanases, cellobiohydrolases, and beta-glucosidases are disclosed in numerous Glycosyl Hydrolase families using the classification according to Henrissat B., 1991, A classification of glycosyl hydrolases based on amino-acid sequence similarities, Biochem. J. 280: 309-316, and Henrissat B., and Bairoch A., 1996, Updating the sequence-based classification of glycosyl hydrolases, Biochem. J. 316: 695-696.

Other cellulolytic enzymes that may be used in the present invention are described in EP 495,257, EP 531,315, EP 531,372, WO 89/09259, WO 94/07998, WO 95/24471, WO 96/11262, WO 96/29397, WO 96/034108, WO 97/14804, WO 98/08940, WO 98/012307, WO 98/13465, WO 98/015619, WO 98/015633, WO 98/028411, WO 99/06574, WO 99/10481, WO 99/025846, WO 99/025847, WO 99/031255, WO 2000/009707, WO 2002/050245, WO 2002/0076792, WO 2002/101078, WO 2003/027306, WO 2003/052054, WO 2003/052055, WO 2003/052056, WO 2003/052057, WO 2003/052118, WO 2004/016760, WO 2004/043980, WO 2004/048592, WO 2005/001065, WO 2005/028636, WO 2005/093050, WO 2005/093073, WO 2006/074005, WO 2006/117432, WO 2007/071818, WO 2007/071820, WO 2008/008070, WO 2008/008793, U.S. Pat. No. 4,435,307, U.S. Pat. No. 5,457,046, U.S. Pat. No. 5,648,263, U.S. Pat. No. 5,686,593, U.S. Pat. No. 5,691,178, U.S. Pat. No. 5,763,254, and U.S. Pat. No. 5,776,757.

In the methods of the present invention, any amylase can be used. In a particular embodiment, the amylase for use according to the invention has alpha-amylase activity, viz. catalyzes the endohydrolysis of 1,4-alpha-glucosidic linkages in oligosaccharides and polysaccharides. Alpha-amylases act, e.g., on starch, glycogen and related polysaccharides and oligosaccharides in a random manner, liberating reducing groups in the alpha-configuration.

In a preferred embodiment the amylase of the invention is an alpha-amylase (systematical name: 1,4-alpha-D-glucan glucanohydrolase). In further embodiments, the amylase of the invention belongs to the EC 3.2.1.-group of amylases, such as EC 3.2.1.1 (alpha-amylase), EC 3.2.1.2 (beta-amylase), EC 3.2.1.3 (glucan 1,4-alpha-glucosidase, amyloglucosidase, or glucoamylase), EC 3.2.1.20 (alpha-glucosidase), EC 3.2.1.60 (glucan 1,4-alpha-maltotetraohydrolase), EC 3.2.1.68 (isoamylase), EC 3.2.1.98 (glucan 1,4-alpha-maltohexosidase), or EC 3.2.1.133 (glucan 1,4-alpha-maltohydrolase).

For purposes of the present invention, preferred amylases are the amylases contained in the following commercial products: BAN, Stainzyme, Termamyl 2X, Termamyl SC, Natalase, and Duramyl (all from Novozymes). Further particular examples of amylases for use according to the invention are the amylases contained in the commercial Validase BAA and Validase HT products (from Valley Research). Still further particular examples of amylases for use according to the invention are the amylases contained in the following commercial products: Clarase, DexLo, GC 262 SP, G-Zyme G990, G-Zyme G995, G-Zyme G997, G-Zyme G998, HTAA, Optimax 7525, Purastar OxAm, Purastar ST, Spezyme AA, Spezyme Alpha, Spezyme BBA, Spezyme Delta AA, Spezyme DBA, Spezyme Ethyl, Spezyme Fred (GC521), Spezyme HPA, Spezyme Extra, and Ultraphlow (all from Genencor); Validase HT340L, Valley Thin 340L (all from Valley Research); Avizyme 1500, Dextro 300 L, Kleistase, Maltazyme, Maxamyl, Thermozyme, Thermatex, Starzyme HT 120 L, Starzyme Super Conc, and Ultraphlo.

In the methods of the present invention, any AA9 polypeptide having cellulolytic enhancing activity can be used.

In a first aspect, the AA9 polypeptide having cellulolytic enhancing activity comprises the following motifs:

[ILMV]-P-X(4,5)-G-X-Y-[ILMV]-X-R-X-[EQ]-X(4)-[HNQ] and [FW]-[TF]-K-[AIV],

wherein X is any amino acid, X(4,5) is any amino acid at 4 or 5 contiguous positions, and X(4) is any amino acid at 4 contiguous positions.

The polypeptide comprising the above-noted motifs may further comprise:

H-X(1,2)-G-P-X(3)-[YW]-[AILMV],

[EQ]-X-Y-X(2)-C-X-[EHQN]-[FILV]-X-[ILV], or

H-X(1,2)-G-P-X(3)-[YW]-[AILMV] and [EQ]-X-Y-X(2)-C-X-[EHQN]-[FILV]-X-[ILV],

wherein X is any amino acid, X(1,2) is any amino acid at 1 position or 2 contiguous positions, X(3) is any amino acid at 3 contiguous positions, and X(2) is any amino acid at 2 contiguous positions. In the above motifs, the accepted IUPAC single letter amino acid abbreviation is employed.

In a preferred aspect, the AA9 polypeptide having cellulolytic enhancing activity further comprises H-X(1,2)-G-P-X(3)-[YW]-[AILMV]. In another preferred aspect, the AA9 polypeptide having cellulolytic enhancing activity further comprises [EQ]-X—Y-X(2)-C-X-[EHQN]-[FILV]-X-[ILV]. In another preferred aspect, the AA9 polypeptide having cellulolytic enhancing activity further comprises H-X(1,2)-G-P-X(3)-[YW]-[AILMV] and [EQ]-X-Y-X(2)-C-X-[EHQN]-[FILV]-X-[ILV].

In a second aspect, the AA9 polypeptide having cellulolytic enhancing activity comprises the following motif:

[ILMV]-P-x(4,5)-G-x-Y-[ILMV]-x-R-x-[EQ]-x(3)-A-[HNQ],

wherein x is any amino acid, x(4,5) is any amino acid at 4 or 5 contiguous positions, and x(3) is any amino acid at 3 contiguous positions. In the above motif, the accepted IUPAC single letter amino acid abbreviation is employed.

Examples of AA9 polypeptides having cellulolytic enhancing activity useful in the methods of the present invention include, but are not limited to, polypeptides having cellulolytic enhancing activity from Thielavia terrestris (WO 2005/074647, WO/2008/148131 and WO 2011/035027); polypeptides having cellulolytic enhancing activity from Thermoascus aurantiacus (WO 2005/074656 and WO 2010/065830); polypeptides having cellulolytic enhancing activity from Trichoderma reesei (WO 2007/089290); and polypeptides having cellulolytic enhancing activity from Myceliophthora thermophila (WO 2009/085935; WO 2009/085859; WO 2009/085864; and WO 2009/085868); polypeptides having cellulolytic enhancing activity from Aspergillus fumigatus (WO 2010/138754); and polypeptides having cellulolytic enhancing activity from Penicillium pinophilum (WO 2011/005867), Thermoascus sp. (WO 2011/039319), Penicillium sp. (WO 2011/041397), and Thermoascus crustaceous (WO 2011/041504).

In one embodiment, the one or more (e.g., several) hemicellulolytic enzymes comprise a commercial hemicellulolytic enzyme preparation. Examples of commercial hemicellulolytic enzyme preparations suitable for use in the present invention include, for example, SHEARZYME™ (Novozymes A/S), CELLIC® HTec (Novozymes A/S), CELLIC® HTec2 (Novozymes A/S), VISCOZYME® (Novozymes A/S), ULTRAFLO® (Novozymes A/S), PULPZYME® HC (Novozymes A/S). MULTIFECT® Xylanase (Genencor), ACCELLERASE® XY (Genencor), ACCELLERASE® XC (Genencor), ECOPULP® TX-200A (AB Enzymes), HSP 6000 Xylanase (DSM), DEPOL™ 333P (Biocatalysts Limit, Wales, UK), DEPOL™ 740L. (Biocatalysts Limit, Wales, UK), and DEPOL™ 762P (Biocatalysts Limit, Wales, UK).

Examples of xylanases useful in the methods of the present invention include, but are not limited to, Aspergillus aculeatus xylanase (GeneSeqP: AAR63790; WO 94/21785); Aspergillus fumigatus xylanases (WO 2006/078256); Penicillium pinophilum (WO 2011/041405); Penicillium sp. (WO 2010/126772); Thielavia terrestris NRRL 8126 (WO 2009/079210); and Trichophaea saccata GH10 (WO 2011/057083).

Examples of beta-xylosidases useful in the methods of the present invention include, but are not limited to, Trichoderma reesei beta-xylosidase (UniProtKB/TrEMBL accession number Q92458); Talaromyces emersonii (SwissProt accession number Q8X212); and Neurospora crassa (SwissProt accession number Q7SOW4).

Examples of acetylxylan esterases useful in the methods of the present invention include, but are not limited to, acetylxylan esterases from Aspergillus aculeatus (WO 2010/108918); Chaetomium globosum (Uniprot accession number Q2GWX4); Chaetomium gracile (GeneSeqP accession number AAB82124); Humicola insolens DSM 1800 (WO 2009/073709); Hypocrea jecorina (WO 2005/001036); Myceliophthora thermophila (WO 2010/014880); Neurospora crassa (UniProt accession number q7s259); Phaeosphaeria nodosum (Uniprot accession number Q0UHJ1); and Thielavia terrestris NRRL 8126 (WO 2009/042846).

Examples of ferulic acid esterases useful in the methods of the present invention include, but are not limited to, Humicola insolens DSM 1800 feruloyl esterase (WO 2009/076122), Neurospora crassa feruloyl esterase (UniProt accession number Q9HGR3), and Neosartorya fischeri feruloyl esterase (UniProt Accession number A1D9T4).

Examples of arabinofuranosidases useful in the methods of the present invention include, but are not limited to, arabinofuranosidases from Aspergillus niger (GeneSeqP accession number AAR94170); Humicola insolens DSM 1800 (WO 2006/114094 and WO 2009/073383); and M. giganteus (WO 2006/114094).

Examples of alpha-glucuronidases useful in the methods of the present invention include, but are not limited to, alpha-glucuronidases from Aspergillus clavatus (UniProt accession number alcc12); Aspergillus fumigatus (SwissProt accession number Q4WW45); Aspergillus niger (Uniprot accession number Q96WX9); Aspergillus terreus (SwissProt accession number Q0CJP9); Humicola insolens (WO 2010/014706); Penicillium aurantiogriseum (WO 2009/068565); Talaromyces emersonii (UniProt accession number Q8X211); and Trichoderma reesei (Uniprot accession number Q99024).

The polypeptides having enzyme activity used in the methods of the present invention may be produced by fermentation of the above-noted microbial strains on a nutrient medium containing suitable carbon and nitrogen sources and inorganic salts, using procedures known in the art (see, e.g., Bennett, J. W. and LaSure, L. (eds.), More Gene Manipulations in Fungi, Academic Press, C A, 1991). Suitable media are available from commercial suppliers or may be prepared according to published compositions (e.g., in catalogues of the American Type Culture Collection). Temperature ranges and other conditions suitable for growth and enzyme production are known in the art (see, e.g., Bailey, J. E., and Ollis, D. F., Biochemical Engineering Fundamentals, McGraw-Hill Book Company, N Y, 1986).

The fermentation can be any method of cultivation of a cell resulting in the expression or isolation of an enzyme or protein. Fermentation may, therefore, be understood as comprising shake flask cultivation, or small- or large-scale fermentation (including continuous, batch, fed-batch, or solid state fermentations) in laboratory or industrial fermentors performed in a suitable medium and under conditions allowing the enzyme to be expressed or isolated. The resulting enzymes produced by the methods described above may be recovered from the fermentation medium and purified by conventional procedures.

Mushroom

A wide variety of mushrooms can benefit from the present invention, and the invention is not limited to any particular mushroom species or strain thereof. In a preferable aspect, the mushroom is selected from the group consisting of Lentinula edodes, Agrocybe aegerita, Auricularia auricular, Auricularia polytricha, Pleurotus ostreatus, Ganoderma Lucidum, Flammulina velutipes, Hericium erinaceus, Hypsizygus marmoreus, Agaricus bisporus, Pleurotus eryngii, Volvariella volvacea, Pleurotus nebrodensis, Pholiota nameko, Tremella fuciformis, and Copyinds comatus. In another preferable aspect, the mushroom is selected from the group consisting of Lentinula edodes, Auricularia auricular, Pleurotus ostreatus, Ganoderma Lucidum, Hericium erinaceus, Agaricus bisporus, Pleurotus eryngii, Volvariella volvacea, Pleurotus nebrodensis, and Copyinds comatus. In another preferable aspect, the mushroom is a grass rot mushroom, such as Agaricus bisporus, Pleurotus eryngii, Volvariella volvacea, Pleurotus nebrodensis, and Copyinds comatus. In another preferable aspect, the mushroom is selected from the group consisting of Lentinula, Agrocybe, Auricularia, Pleurotus, Ganoderma, Flammulina, Hericium, Hypsizygus, Agaricus, Volvariella, Pholiota, Tremella, and Copyinds. In another preferable aspect, the mushroom is selected from the group consisting of Volvariella, Flammulina, Agaricus and Pleurotus. In another preferable aspect, the mushroom is selected from the group consisting of Volvariella volvacea, Flammulina velutipes, Agaricus bisporus and Pleurotus eryngii.

The invention is further defined in the following paragraphs:

[1]. A method for cultivating mushrooms, comprising:

(a) providing a culture material, and

(b) mixing the culture material with an enzyme for degrading or converting cellulosic material; more preferably, an enzyme having cellulolytic activity and/or hemicellulolytic activity.

[2]. The method of paragraph 1, wherein the enzyme comprises a cellulase, and optionally one or more (e.g., several) enzymes selected from the group consisting of a AA9 polypeptide having cellulolytic enhancing activity, a hemicellulase, an amylase, an esterase, an expansin, a catalase, a laccase, a ligninolytic enzyme, a pectinase, a peroxidase, a protease, and a swollenin.

[3]. The method of paragraph 2, wherein the enzyme is mixed with the culture material in an amount of about 0.005 to about 100 mg, preferably about 0.01 to about 50 mg, more preferably about 0.05 to about 25 mg, more preferably about 0.1 to about 20 mg, more preferably about 0.1 to about 15 mg, even more preferably about 0.1 to about 10 mg, and most preferably about 0.5 to about 10 mg enzyme, per g of culture material.

[4]. The method of any of paragraphs 1-3, wherein the enzyme comprises cellulase and hemicellulase, preferably an endoglucanase, a cellobiohydrolase, a beta-glucosidase and a xylanase.

[5]. The method of paragraph 4, wherein the cellulase is one or more (e.g., several) enzymes selected from the group consisting of an endoglucanase, a cellobiohydrolase, and a beta-glucosidase.

[6]. The method of paragraph 4 or 5, wherein the cellulase is mixed with the culture material in an amount of about 0.005 to about 50 mg, preferably about 0.01 to about 40 mg, more preferably about 0.05 to about 25 mg, more preferably about 0.1 to about 20 mg, more preferably about 0.25 to about 15 mg, even more preferably about 0.5 to about 10 mg, and most preferably about 0.5 to about 5 mg cellulase, per g of culture material.

[7]. The method of paragraph 4, wherein the hemicellulase is one or more (e.g., several) enzymes selected from the group consisting of an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase.

[9]. The method of any of paragraphs 4-8, wherein the hemicellulase is mixed with the culture material in an amount of about 0.001 to about 50 mg, preferably about 0.005 to about 40 mg, more preferably about 0.01 to about 25 mg, more preferably about 0.05 to about 20 mg, more preferably about 0.05 to about 15 mg, even more preferably about 0.075 to about 10 mg, and most preferably about 0.1 to about 5 mg hemicellulase, per g of culture material.

[10]. The method of any of paragraphs 1-9, wherein the culture material comprises cellulosic material selected from the group consisting of agricultural residue, herbaceous material, municipal solid waste, pulp and paper mill residue, waste paper, bran and wood; preferably, arundo, bagasse, bamboo, corn cob, corn fiber, corn stover, miscanthus, orange peel, rice straw, switchgrass, wheat straw, eucalyptus, fir, pine, poplar, spruce, willow, algal cellulose, bacterial cellulose, cotton waste, cotton linter, filter paper, saw dust, kernel of grains and hull of cotton seed.

[11]. The method of paragraph 10, wherein the culture material further comprises manure, starch, gypsum, sugar, urea, calcium superphosphate, calcium carbonate and/or lime.

[12]. The method of any of paragraphs 1-11, comprising

(a) providing a culture material,

(b) mixing the culture material with an enzyme composition comprising cellulase and hemicellulase.

[13]. The method of any of paragraphs 1-11, comprising

(a) providing a culture material,

(b) mixing the culture material with cellulase,

(c) mixing the culture material with hemicellulase,

wherein step (b) and step (c) is carried out simultaneously or sequentially.

[14]. The method of paragraph 12 or 13, wherein hemicellulase and cellulase are in an amount of about 0.005 to about 1.0 g, preferably about 0.005 to about 0.75 g, more preferably about 0.01 to about 0.75 g, more preferably about 0.05 to about 0.5 g, more preferably about 0.075 to about 0.5 g, even more preferably about 0.1 to about 0.5 g, and most preferably about 0.1 to about 0.45 g hemicellulase, per g of cellulase.

[15]. The method of any of paragraphs 1-14, further comprising

subjecting the mixed culture material to composting;

optionally, further comprising subjecting the composted culture material to a second composting.

[16]. The method of any of paragraphs 1-15, further comprising

inoculating the culture material with mushroom mycelia and allowing the mushroom mycelia to develop into mushroom; and optionally recovering the mushroom.

[17]. The method of any of paragraphs 1-16, wherein the mushroom is selected from the group consisting of Lentinula edodes, Agrocybe aegerita, Auricularia auricular, Auricularia polytricha, Pleurotus ostreatus, Ganoderma Lucidum, Flammulina velutipes, Hericium erinaceus, Hypsizygus marmoreus, Agaricus bisporus, Pleurotus eryngii, Volvariella volvacea, Pleurotus nebrodensis, Pholiota nameko, Tremella fuciformis, and Copyinds comatus.

[18]. A method for improving the yield of mushrooms and/or biological efficiency of mushroom cultivation, comprising:

(a) providing a culture material, and

(b) mixing the culture material with an enzyme for degrading or converting cellulosic material, preferably, an enzyme having cellulolytic activity and/or hemicellulolytic activity.

[19]. The method of paragraph 18, wherein the enzyme comprises a cellulase, and optionally one or more (e.g., several) enzymes selected from the group consisting of a AA9 polypeptide having cellulolytic enhancing activity, a hemicellulase, an amylase, an esterase, an expansin, a catalase, a laccase, a ligninolytic enzyme, a pectinase, a peroxidase, a protease, and a swollenin.

[20]. The method of paragraph 19, wherein the enzyme is mixed with the culture material in an amount of about 0.005 to about 100 mg, preferably about 0.01 to about 50 mg, more preferably about 0.05 to about 25 mg, more preferably about 0.1 to about 20 mg, more preferably about 0.1 to about 15 mg, even more preferably about 0.1 to about 10 mg, and most preferably about 0.5 to about 10 mg enzyme, per g of the culture material.

[21]. The method of any of paragraphs 18-20, wherein the enzyme comprises cellulase and hemicellulase, preferably an endoglucanase, a cellobiohydrolase, a beta-glucosidase and a xylanase.

[22]. The method of paragraph 21, wherein the cellulase is one or more (e.g., several) enzymes selected from the group consisting of an endoglucanase, a cellobiohydrolase, and a beta-glucosidase.

[23]. The method of paragraph 21 or 22, wherein the cellulase is mixed with the culture material in an amount of about 0.005 to about 50 mg, preferably about 0.01 to about 40 mg, more preferably about 0.05 to about 25 mg, more preferably about 0.1 to about 20 mg, more preferably about 0.25 to about 15 mg, even more preferably about 0.5 to about 10 mg, and most preferably about 0.5 to about 5 mg cellulase per g of the culture material.

[24]. The method of paragraph 21, wherein the hemicellulase is one or more (e.g., several) enzymes selected from the group consisting of an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase.

[25]. The method of any of paragraphs 21-24, wherein the hemicellulase is mixed with the culture material in an amount of about 0.001 to about 50 mg, preferably about 0.005 to about 40 mg, more preferably about 0.01 to about 25 mg, more preferably about 0.05 to about 20 mg, more preferably about 0.05 to about 15 mg, even more preferably about 0.075 to about 10 mg, and most preferably about 0.1 to about 5 mg hemicellulase per g of the culture material.

[27]. The method of any of paragraphs 17-26, wherein the culture material comprises the cellulosic material which is selected from the group consisting of agricultural residue, herbaceous material, municipal solid waste, pulp and paper mill residue, waste paper, bran and wood; preferably, arundo, bagasse, bamboo, corn cob, corn fiber, corn stover, miscanthus, orange peel, rice straw, switchgrass, wheat straw, eucalyptus, fir, pine, poplar, spruce, willow, algal cellulose, bacterial cellulose, cotton waste, cotton linter, filter paper, saw dust, kernel of grains, and hull of cotton seed.

[28]. The method of paragraph 27, wherein the culture material further comprises manure, starch, gypsum, sugar, urea, calcium superphosphate, calcium carbonate and/or lime.

[29]. The method of any of paragraphs 18-28, comprising

(a) providing a culture material,

(b) mixing the culture material with an enzyme composition comprising cellulase and hemicellulase.

[30]. The method of any of paragraphs 18-28, comprising

(a) providing a culture material,

(b) mixing the culture material with cellulase,

(c) mixing the culture material with hemicellulase,

wherein step (b) and step (c) is carried out simultaneously or sequentially.

[31]. The method of paragraph 29 or 30, wherein hemicellulase and cellulase are in an amount of about 0.005 to about 1.0 g, preferably about 0.005 to about 0.75 g, more preferably about 0.01 to about 0.75 g, more preferably about 0.05 to about 0.5 g, more preferably about 0.075 to about 0.5 g, even more preferably about 0.1 to about 0.5 g, and most preferably about 0.1 to about 0.45 g hemicellulase, per g of cellulase.

[31]. The method of any of paragraphs 18-31, further comprising

subjecting the mixed culture material to composting;

optionally, further comprising subjecting the composted culture material to a second composting.

[32]. The method of any of paragraphs 18-31, further comprising

inoculating the culture material with mushroom mycelia and allowing the mushroom mycelia to develop into mushroom; and optionally recovering the mushroom.

[33]. The method of any of paragraphs 18-32, wherein the mushroom is selected from the group consisting of Lentinula edodes, Agrocybe aegerita, Auricularia auricular, Auricularia polytricha, Pleurotus ostreatus, Ganoderma Lucidum, Flammulina velutipes, Hericium erinaceus, Hypsizygus marmoreus, Agaricus bisporus, Pleurotus eryngii, Volvariella volvacea, Pleurotus nebrodensis, Pholiota nameko, Tremella fuciformis, and Copyinds comatus.

[34]. An enzyme composition for mushroom cultivation, comprising an enzyme for degrading or converting cellulosic material; preferably comprising one or more (e.g., several) enzymes selected from the group consisting of a cellulase, a AA9 polypeptide having cellulolytic enhancing activity, a hemicellulase, an amylase, an esterase, an expansin, a catalase, a laccase, a ligninolytic enzyme, a pectinase, a peroxidase, a protease, and a swollenin.

[35]. The enzyme composition of paragraph 34, wherein the enzyme composition comprises cellulase and hemicellulase, preferably an endoglucanase, a cellobiohydrolase, a beta-glucosidase and a xylanase.

[36]. The enzyme composition of paragraph 34 or 35, wherein the cellulase is one or more (e.g., several) enzymes selected from the group consisting of an endoglucanase, a cellobiohydrolase, and a beta-glucosidase.

[37]. The enzyme composition of any of paragraphs 34-36, wherein the hemicellulase is one or more (e.g., several) enzymes selected from the group consisting of an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a xylanase, and a xylosidase.

[38]. The enzyme composition of any of paragraphs 34-37, wherein the hemicellulase to the cellulase is in an amount of about 0.005 to about 1.0 g, preferably about 0.005 to about 0.75 g, more preferably about 0.01 to about 0.75 g, more preferably about 0.05 to about 0.5 g, more preferably about 0.075 to about 0.5 g, even more preferably about 0.1 to about 0.5 g, and most preferably about 0.1 to about 0.45 g hemicellulase, per g of cellulase.

[39]. The enzyme composition of any of paragraphs 34-38, wherein the mushroom is selected from the group consisting of Lentinula edodes, Agrocybe aegerita, Auricularia auricular, Auricularia polytricha, Pleurotus ostreatus, Ganoderma Lucidum, Flammulina velutipes, Hericium erinaceus, Hypsizygus marmoreus, Agaricus bisporus, Pleurotus eryngii, Volvariella volvacea, Pleurotus nebrodensis, Pholiota nameko, Tremella fuciformis, and Copyinds comatus.

[40]. Use of the enzyme composition of any of paragraphs 34-39 for mushroom cultivation.

[41]. Use of the enzyme composition of any of paragraphs 34-39 for improvement of yield of mushroom and/or biological efficiency of mushroom cultivation.

[42]. Use of a cellulase and a hemicellulose for mushroom cultivation.

[43]. Use of a cellulase and a hemicellulose for improvement of yield of mushroom and/or biological efficiency of mushroom cultivation.

The present invention is further described by the following examples that should not be construed as limiting the scope of the invention.

### EXAMPLES

### Enzymes

Cellic® Ctec2, a cellulase product commercially available from Novozymes A/S;

Celluclean® Classic 700T, a cellulase product commercially available from Novozymes A/S;

Cellic® Htec2, a hemicellulase product commercially available from Novozymes A/S.

### Example 1

### Yield Improvement of Volvariella volvacea Mushroom by Addition of Enzymes to Substrates

Methods and Steps

1. Drying of Raw Material

Cotton waste (available from a textile factory) was exposed to sunlight for 3-4 days to prevent bacterial or fungal contamination.

2. Mixing

92% by weight of cotton waste and 8% by weight of lime was mixed (carbon:nitrogen of about 23:1) and water was added thereto, resulting a culture material with water content of about 75% and pH of about 8-8.5.

3. First Composting

Seven piles of mixed culture material were made with 50 kg dry weight each, numbered as 1-7. Relevant enzymes were added to group “2-4” according to the Table 1 and then mixed thoroughly. pH was adjusted to 8 with lime. The piles were composted for one day, the temperature was kept at 55-60° C., pH was adjusted to 8. Then the piles were turned and relevant enzymes were added to group “5-7” according to Table 1, pH was adjusted to 8 and the temperature was kept at 50-60° C. Group “1” was taken as control. Water content of the piles was kept at 65%-75%.

TABLE 1
Enzyme composition during mushroom cultivation

| | Cellic ® | Celluclean ® | Cellic ® | |
|---|---|---|---|---|
| Groups No. | Ctec 2 | Classic 700T | Htec 2 | Addition time |
| No. 1 (control) | — | — | — | — |
| No. 2 | 0.125% | 0.125% | 0.0625% | At the beginning of first composting |
| No. 3 | 0.5% | 0.5% | 0.25% | At the beginning of first composting |
| No. 4 | 1% | 1% | 0.5% | At the beginning of first composting |
| No. 5 | 0.125% | 0.125% | 0.0625% | At the end of first composting |
| No. 6 | 0.5% | 0.5% | 0.25% | At the end of first composting |
| No. 7 | 1% | 1% | 0.5% | At the end of first composting |

4. Feeding and Spraying of Culture Material

The culture material was sprayed onto cultivation bed, with thickness of 8-10 cm. Each group/treatment took up a bed shelf including 3 beds. 1×1.5 m² was used as one bed with around 16.67 kg dry weight of culture material.

5. Second Composting

The cultivation substrate was subjected to 65-70° C. with burning coal for 1 day.

6. Cooling

Culture material temperature was subjected to drop to 36-38° C. The resulted pH was about pH 7.5-7.8 and water content was about 68%.

7. Inoculation

The composted culture material (or cultivation substrate) was inoculated with propagated Volvariella volvacea seeds. The inoculation amount was about 1.5% of dry weight of culture material, which is around 0.13 kg/m².

8. Mycelium Growth

Room temperature was kept at 28-32° C., and the substrate temperature was kept at 33-36° C., water content of cultivation substrate at around 70%, air humidity at 80%, pH7.8-8.0. It took around 4 days for the mycelium to grow to the bottom layer of cultivation substrate. Then cultivation room was ventilated to dry the surface of the substrate. After that, water was sprayed to the substrate until water drops oozing from the bottom layer and then cultivation room was ventilated again. The temperature was kept at 28° C. within 5-6 hours after water spraying, and the substrate water content was controlled back to around 70%.

9. Mushroom Growth The substrate temperature was kept at 28-33° C. and water content at 70%-75%, air humidity at 90%, pH7.0-8.0. The surface temperature of the material was kept at 30-32° C. 500-1000 lx light was needed during this period of time.

### Conclusion

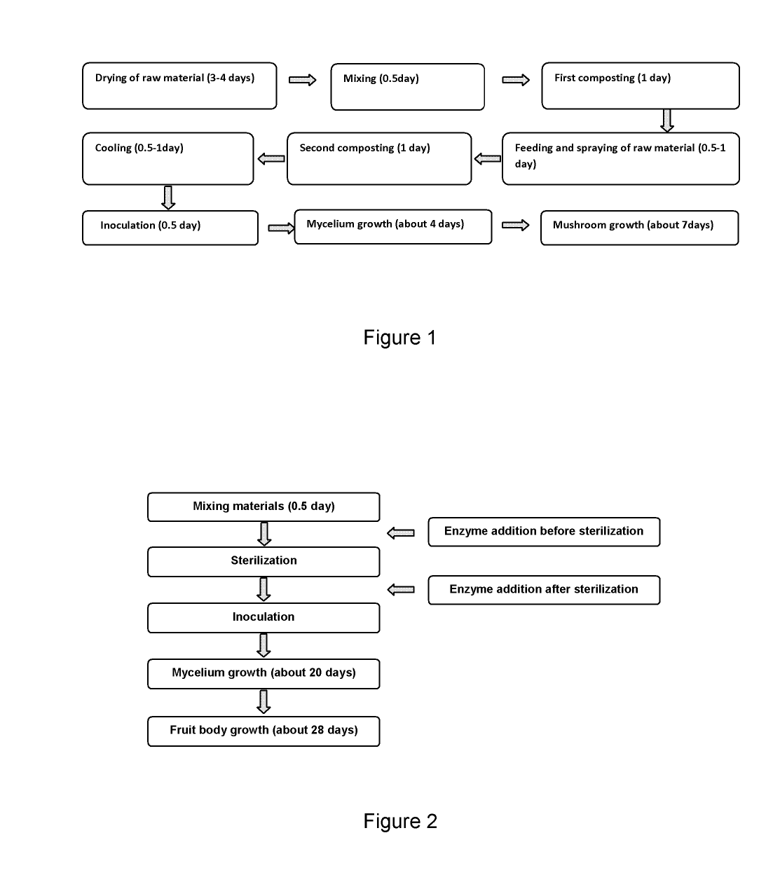

The flowchart of cultivating process is shown in FIG. 1. Production yield is shown in Table 2. From table 2, it can be seen that the addition of enzyme improves the production yield of mushroom. It can also be seen that the addition of enzyme improves biological efficiency.

TABLE 2
Production yield of mushroom with enzyme addition

| | Yields of | | |
|---|---|---|---|
| | fresh mushroom | The biological | Yield |
| Groups No. | (kg/treatment) | efficiency* (%) | improvement (%) |
| No. 1 (control) | 11.5 | 17.24 | 0% |
| No. 2 | 13.5 | 20.24 | 17% |
| No. 3 | 13.25 | 19.87 | 15% |
| No. 4 | 12.75 | 19.12 | 10% |
| No. 5 | 13 | 19.49 | 13% |
| No. 6 | 12.5 | 18.74 | 8% |
| No. 7 | 12 | 17.99 | 4% |

*The biological efficiency = Yields of fresh mushroom (kg)/dry weight of culture material (kg) × 100%.

### Example 2

### Yield Improvement of Flammulina velutipes Mushroom by Addition of Enzymes to Cultivation Substrates Before Sterilization

Methods and Steps

1. Prepare Cultivation Substrates

Materials comprising 40% cottonseed hulls, 15% sawdust, 20% corn cob, 20% bran, 3% corn meal, 1% gesso powder and 1% lime powder were mixed. Then water was added into these materials and the moisture content was made to 60-65%. After thoroughly mixing, the cultivation substrates were divided into several bags with 175 g dry substance each.

2. Enzyme Treatment and Sterilization

For the test with enzyme treatment before sterilization, relevant enzymes were added with water to group “2” according to Table 3 and mixed thoroughly, and then the materials were incubated at 50° C., pH 5.0 for 3 days with stirring every 12 hours. The pH was adjusted to 8 with lime after treatment, and moisture content was kept as 60-65%. Then the enzyme treated substrates were sterilized by an autoclave. Group of “1” is taken as control without enzyme treatment.

TABLE 3
Composition of enzymes for enoki mushroom cultivation.

| Group | | | Alpha- | |
|---|---|---|---|---|
| No. | Cellulase | Hemicellulase | amylase | Dosing point |
| 1 | — | — | — | — |
| 2 | 1.32 mg/g material | 0.345 mg/g | 0.06 mg/g | Before |
| | | material | material | sterilization |

3. Inoculation

15-20 g solid Enoki mushroom (Flammulina velutipes) seeds were inoculated to each bag of cultivation substrates.

4. Mycelium Growth

The mycelium of enoki mushroom were growed in dark under the conditions at temperature of 20-22° C., air humidity of 60-65%, CO₂ concentration under 4000 mL/m³. It took about 20 days for the mycelium to grow to the bottom layer of cultivation substrate. Then cultivation room was ventilated to dry the surface of the substrate. The fungi were scratched off the surface layer, once mycelium was full of the bag.

5. Fruit Body Growth

For fruit body growth, the enoki mushroom was cultured under temperature of 12-15° C., air humidity of 90-95% and light of 600-800 LX for 8 to 10 days. For the following 7 to 8 days, temperature was gradually decreased to 3-5° C., air humidity and CO₂ concentration were kept at 80-90% and 2000-2500 mL/m³, respectively. After the fruit body came out, the conditions were controlled with temperature of 8-10° C., air humidity of 90-95% and CO₂ concentration of 5000-6000 mL/m³.

### Conclusion

The process flowchart of enoki mushroom cultivation is shown in FIG. 2. Yield performance with/without enzyme treatment is shown in Table 4. It can be seen that the group “2” with addition of enzyme before medium sterilization improve the production yield of enoki mushroom and the biological efficiency of mushroom cultivation.

TABLE 4
Production yield of enoki mushroom with enzyme addition.

| | Yield of fresh | The biological | |
|---|---|---|---|
| Group No. | mushroom (g) | efficiency* (%) | Yield improvement (%) |
| 1 | 86.12 | 49.21% | 0% |
| 2 | 95.28 | 54.44% | 10.63% |

*The biological efficiency = Yields of fresh mushroom (g)/dry weight of culture material (g) × 100%.

### Example 3

### Yield Improvement of Flammulina velutipes Mushroom by Addition of Enzymes to Cultivation Substrates after Sterilization

Methods and Steps

1. Prepare Cultivation Substrates

Materials comprising 40% cottonseed hulls, 15% sawdust, 20% corn cob, 20% bran, 3% corn meal, 1% gesso powder and 1% lime powder were mixed. Then water was added into these materials and the moisture content was made to 60-65%. After thoroughly mixing, the cultivation substrates were divided into several bottles with 175 g dry substance each.

2. Enzyme Treatment and Sterilization

For the group as “2” according to Table 5 with enzyme treatment after sterilization, the moisture content of substrates was first kept 45% for autoclave treatment, and then relevant enzymes were added with sterilized water to the substrates after autoclave treatment to make the final moisture content to 60-65%. Group of “1” is taken as control without enzyme treatment.

TABLE 5
Composition of enzymes for enoki mushroom cultivation.

| Group No. | Cellulase | Hemicellulase | Alpha-amylase | Dosing point |
|---|---|---|---|---|
| 1 | — | — | — | — |
| 2 | 1.32 mg/g | 0.345 mg/g | 0.06 mg/g | After |
| | material | material | material | sterilization |

3. Inoculation

15-20 g solid Enoki mushroom (Flammulina velutipes) seeds were inoculated to each bottle of cultivation substrates.

4. Mycelium Growth

The mycelium of enoki mushroom were growed in dark under the conditions at temperature of 20-22° C., air humidity of 60-65%, CO₂ concentration under 4000 mL/m³. It took about 20 days for the mycelium to grow to the bottom layer of cultivation substrate. Then cultivation room was ventilated to dry the surface of the substrate. The fungi were scratched off the surface layer, once mycelium was full of the bag.

5. Fruit Body Growth

For fruit body growth, the enoki mushroom was cultured under temperature of 12-15° C., air humidity of 90-95% and light of 600-800 LX for 8 to 10 days. For the following 7 to 8 days, temperature was gradually decreased to 3-5° C., air humidity and CO₂ concentration were kept at 80-90% and 2000-2500 mL/m³, respectively. After the fruit body came out, the conditions were controlled with temperature of 8-10° C., air humidity of 90-95% and CO₂ concentration of 5000-6000 mL/m³.

### Conclusion

The process flowchart of enoki mushroom cultivation is shown in FIG. 2. Yield performance with/without enzyme treatment is shown in Table 6. It can be seen that the group “2” with addition of enzyme after medium sterilization improve the production yield of enoki mushroom and the biological efficiency of mushroom cultivation.

TABLE 6
Production yield of enoki mushroom with enzyme addition.

| | Yield of fresh | The biological | |
|---|---|---|---|
| Group No. | mushroom (g) | efficiency* (%) | Yield improvement (%) |
| 1 | 125.85 | 55.32% | 0% |
| 2 | 137.42 | 60.41% | 9.19% |

*The biological efficiency = Yields of fresh mushroom (g)/dry weight of culture material (g) × 100%.

### Example 4

### Yield Improvement of Agaricus bisporus Mushroom by Addition of Enzymes to Substrates

Methods and Steps

1. Raw Material Preparation

Rice stock and cow dung were prewet for 3-4 days and 6-8 days respectively. Materials with the composition of 54% by weight of rice stock, 36% by weight of cow dung, 3.5% by weight of urea, 3.5% by weight of calcium superphosphate, 1.5% by weight of gypsum and 1.5% by weight of lime were then mixed and adjusted to 65% water content.

2. First Composting

Four piles of mixed culture material were made with 50 kg dry weight each, numbered as 1→4. Each pile was hide-lifted and adjusted to pH 7, 65% water content at the 6^{th} day, the 11^{th} day and the 16^{th} day respectively. At the 18^{th} day, the piles were turned and relevant enzymes were added to group “2-4” according to Table 7, pH was adjusted to 7 and water content was kept at 65%. Group “1” was taken as control.

TABLE 7
Enzyme composition during mushroom cultivation

| Groups | | | |
|---|---|---|---|
| No. | Cellulase | Hemicellulase | Addition time |
| No. 1 | — | — | — |
| (control) | | | |
| No. 2 | 0.36 mg/g material | 0.14 mg/g material | At the end of first |
| | | | composting |
| No. 3 | 1.45 mg/g material | 0.58 mg/g material | At the end of first |
| | | | composting |
| No. 4 | 2.90 mg/g material | 1.15 mg/g material | At the end of first |
| | | | composting |

3. Feeding and Spraying of Culture Material

The culture material was sprayed onto cultivation bed. Each group/treatment took up a bed shelf including 3 beds. 1×0.5 m² was used as one bed.

4. Second Composting

The cultivation substrate was subjected to 60-65° C. for 1 day. The temperature was then decreased to 48-52° C. and kept for 5-7 days.

5. Cooling

Culture material temperature was subjected to drop to 30° C. 6. Inoculation

The composted culture material (or cultivation substrate) was inoculated with propagated Agaricus bisporus seeds. The inoculation amount was about 1.5% of dry weight of culture material, which is around 0.13 kg/m².

7. Mycelium Growth

Room temperature was kept at 25° C., water content of cultivation substrate at around 70-75% for around 21 days. The beds were then covered with soil for around 3 cm thickness. Water was then added to adjust the covering soil to 18-10% water content and kept for 3-4 days.

8. Mushroom Growth

The substrate temperature was kept at 15-16° C., air humidity at 90%. Water was poured to the beds starting from the point when mycelium grew to the height of 1 cm distance to the surface of the covered soil. 3-4 kg/cm² water in total to be poured within 6 days. Then high quality water was poured to the beds starting from the point when the mushroom bud grew to the size of soybean. 2.5-3 kg/cm² water in total to be poured within another 6 days.

9. Second Tide Mushroom Growth

Second tide mushroom was collected in another 11 days.

10. Third Tide Mushroom Growth

Third tide mushroom was collected in another 16 days.

### Conclusion

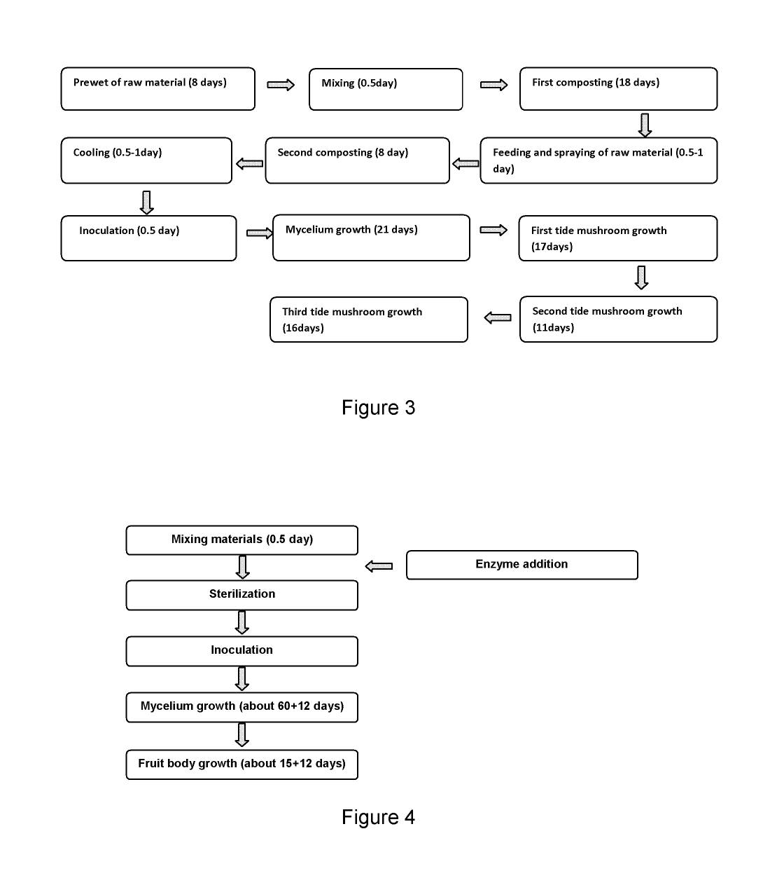

The flowchart of cultivating process is shown in FIG. 3. Production yield is shown in Table 8. From table 8, it can be seen that the addition of enzyme improves the production yield of mushroom. It can also be seen that the addition of enzyme improves biological efficiency.

TABLE 8
Production yield of mushroom with enzyme addition

| | Yields of fresh | The biological | |
|---|---|---|---|
| | mushroom | efficiency* | Yield improvement |
| Groups No. | (g/treatment) | (%) | (%) |
| No. 1 (control) | 1715.8 | 33.51 | 0% |
| No. 2 | 1850.2 | 36.14 | 8% |
| No. 3 | 1765.51 | 34.48 | 3% |
| No. 4 | 1929.78 | 37.69 | 12% |

*The biological efficiency = Yields of fresh mushroom (kg)/dry weight of culture material (kg) × 100%.

### Example 5

### Yield Improvement of Pleurotus eryngii Mushroom by Addition of Enzymes to Cultivation Substrates

Methods and Steps 1. Prepare Cultivation Substrates

Materials comprising 35% by weight sawdust, 35% by weight corncob, 20% by weight bran, 5% by weight corn meal, 4% by weight soy bean meal and 1% by weight lime powder were mixed. Then water was added into these materials and the moisture content was made to 66-68%. pH was kept at 6.5. After thoroughly mixing, the cultivation substrates were divided into several bags with 550 g dry substance each.

2. Enzyme Treatment and Sterilization

For the test groups with enzyme treatment before sterilization, relevant enzymes were added with water to group “2-4” according to Table 9 and mixed thoroughly, and then the materials were incubated at 40° C. for 24 hours. Then all the substrates were sterilized by an autoclave. Group “1” is taken as control without enzyme treatment.

TABLE 9
Composition of enzymes Pleurotus eryngii mushroom cultivation.

| Group | | | |
|---|---|---|---|
| No. | Cellulase | Hemicellulase | Dosing point |
| 1 | — | — | — |
| 2 | 0.55 mg/g material | 0.14 mg/g material | Before sterilization |
| 3 | 2.20 mg/g material | 0.58 mg/g material | Before sterilization |
| 4 | 8.80 mg/g material | 2.30 mg/g material | Before sterilization |

3. Inoculation

10 g solid Pleurotus eryngii seeds were inoculated to each bag of cultivation substrates.

4. Mycelium Growth

The mycelium of Pleurotus eryngii mushroom were grew in dark under the conditions at temperature of 25° C., air humidity of 60-65%, 10-16 m³/h ventilation. It took about 60 days for the mycelium to grow to the bottom layer of cultivation substrate. The bags were kept for another 12 days for postripeness.

5. Fruit Body Growth

For fruit body growth, the Pleurotus eryngii was cultured under temperature of 13° C., air humidity of 93%, moderate ventilation to keep the CO₂ concentration below 0.1% and light of 500-800 LX for 15 days. After the fruit body came out, extra buds were picked up and only 1-2 Pleurotus eryngii buds were retained for each bag. The conditions were then controlled with temperature of 15-17° C., air humidity of 90-95% at the beginning and 85-90% for growth in later stage, and light of 500-1000 LX.

### CONCLUSION

The process flowchart of Pleurotus eryngii cultivation is shown in FIG. 4. Yield performance with/without enzyme treatment is shown in Table 10. It can be seen that the group “2” and “3” with addition of enzyme improves the production yield of Pleurotus eryngii significantly.

TABLE 10
Production yield of Pleurotus eryngii with enzyme addition.

| | Yield of fresh | The biological | |
|---|---|---|---|
| Group No. | mushroom (g) | efficiency* (%) | Yield improvement (%) |
| 1 | 308.7 ± 47.7 | 56.1% | 0% |
| 2 | 351.9 ± 30.1 | 64.0% | 14.0% |
| 3 | 376.2 ± 21.3 | 68.4% | 21.9% |
| 4 | 309.6 ± 45.5 | 56.3% | 0.3% |

*The biological efficiency = Yields of fresh mushroom (g)/dry weight of culture material (g) × 100%.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A method for cultivating mushrooms, comprising:
(a) providing a culture material, and
(b) mixing the culture material with an enzyme for degrading or converting cellulosic material.

2. The method of claim 1, wherein the enzyme comprises a cellulase and optionally one or more enzymes selected from the group consisting of a AA9 polypeptide having cellulolytic enhancing activity, a hemicellulase, an amylase, an esterase, an expansin, a catalase, a laccase, a ligninolytic enzyme, a pectinase, a peroxidase, a protease, and a swollenin.

3. The method of claim 1, wherein the enzyme is mixed with the culture material in an amount of about 0.005 to about 100 mg enzyme, per g of culture material.

4. The method of claim 1, wherein the culture material comprises cellulosic material selected from the group consisting of agricultural residue, herbaceous material, municipal solid waste, pulp and paper mill residue, waste paper, bran and wood; preferably, arundo, bagasse, bamboo, corn cob, corn fiber, corn stover, miscanthus, orange peel, rice straw, switchgrass, wheat straw, eucalyptus, fir, pine, poplar, spruce, willow, algal cellulose, bacterial cellulose, cotton waste, cotton linter, filter paper, saw dust, kernel of grains and hull of cotton seed; more preferably, the culture material further comprises manure, starch, gypsum, sugar, urea, calcium superphosphate, calcium carbonate and/or lime.

5. The method of claim 1, wherein the mushroom is selected from the group consisting of Lentinula edodes, Agrocybe aegerita, Auricularia auricular, Auricularia polytricha, Pleurotus ostreatus, Ganoderma Lucidum, Flammulina velutipes, Hericium erinaceus, Hypsizygus marmoreus, Agaricus bisporus, Pleurotus eryngii, Volvariella volvacea, Pleurotus nebrodensis, Pholiota nameko, Tremella fuciformis, and Copyinds comatus.

6. The method of claim 1, comprising
(a) providing a culture material,
(b) mixing the culture material with a cellulase,
(c) mixing the culture material with a hemicellulase,
wherein step (b) and step (c) is carried out simultaneously or sequentially.

7. A method for improving the yield of mushrooms and/or biological efficiency of mushroom cultivation, comprising:
(a) providing a culture material, and
(b) mixing the culture material with an enzyme for degrading or converting cellulosic material.

8. The method of claim 7, wherein the enzyme comprises a cellulase and optionally one or more enzymes selected from the group consisting of a AA9 polypeptide having cellulolytic enhancing activity, a hemicellulase, an amylase, an esterase, an expansin, a catalase, a laccase, a ligninolytic enzyme, a pectinase, a peroxidase, a protease, and a swollenin.

9. The method of claim 7, wherein enzyme is mixed with the culture material in an amount of about 0.005 to about 100 mg enzyme, per g of the culture material.

10. The method of claim 7, comprising
(a) providing a culture material,
(b) mixing the culture material with a cellulase,
(c) mixing the culture material with a hemicellulase,
wherein step (b) and step (c) is carried out simultaneously or sequentially.

11. An enzyme composition for mushroom cultivation, comprising an enzyme for degrading or converting cellulosic material.

12. The enzyme composition of claim 11, wherein the enzyme composition comprises cellulase and hemicellulase in an amount of about 0.005 to about 1.0 g.

13.-15. (canceled)
